(19) United States
(12) Patent Application Publication
(10) Pub. No.: US 2018/0174671 A1
(43) Pub. Date: Jun. 21, 2018

(54) COGNITIVE ADAPTATIONS FOR WELL-BEING MANAGEMENT

(71) Applicant: International Business Machines Corporation, Armonk, NY (US)

(72) Inventors: Luis Carlos Cruz Huertas, San Pedro (CR); Rick A. Hamilton, II, Charlottesville, VA (US); Hari K. Narasimhamurthy, Chantilly, VA (US); Edgar Adolfo Zamora Duran, Santo Domingo (CR)

(21) Appl. No.: 15/836,980

(22) Filed: Dec. 11, 2017

Related U.S. Application Data
(63) Continuation of application No. 15/380,005, filed on Dec. 15, 2016.

(51) Int. Cl. G06N 99/00

(52) U.S. Cl. CPC G16H 15/00; G06N 99/005; G16H 10/60

(57) ABSTRACT

Disclosed aspects relate to cognitive adaptations for well-being management in a living environment. A set of sensor-derived data for the living environment may be ingested. The ingestion of a set of sensor-derived data may occur using a set of micro-cognitive modules. The set of sensor-derived data may be analyzed using a machine learning technique. The set of sensor-derived data may be analyzed to detect an anomalous event related to the living environment. The anomalous event may be detected based on the set of sensor-derived data. An anomalous event response action may be performed in response to detecting the anomalous event.

### BACKGROUND

This disclosure relates generally to computer systems and, more particularly, relates to cognitive adaptations for well-being management. Well-being in a living environment may be desired to be monitored on a regular basis. The number of individuals in independent living environments may be increasing. As the number of people in independent living environments increases, the need for cognitive adaptations such as cognitive systems or micro cognitive systems for well-being management in a living environment may also increase.

### SUMMARY

Aspects of the disclosure relate to cognitive adaptations for well-being management in a living environment. Disclosed aspects may utilize analytics and measurements from micro-cognitive systems to determine an anomalous state, and thereafter perform an action once the anomalous state is detected. Inputs from a plurality of micro cognitive systems may be received. The micro-cognitive systems may be configured to self-learn, identify a set of behavior patterns of an individual, and to trigger an alarm parameter in response to a pattern mismatch. The inputs received from the plurality of micro cognitive systems may be integrated to form integrated data by a central processing system. The integrated data may be analyzed to identify behavioral data and alarm parameters. Behavioral norms for a subject may be established using the behavioral data and the identified alarm parameters. Predetermined criteria may be used to determine whether an anomalous event has been detected, and a response action may be performed in response to determining the anomalous event.

Disclosed aspects relate to cognitive adaptations for well-being management in a living environment. A set of sensor-derived data for the living environment may be ingested. The ingestion of a set of sensor-derived data may occur using a set of micro-cognitive modules. The set of sensor-derived data may be analyzed using a machine learning technique. The set of sensor-derived data may be analyzed to detect an anomalous event related to the living environment. The anomalous event may be detected based on the set of sensor-derived data. An anomalous event response action may be performed in response to detecting the anomalous event.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts a high-level block diagram of a computer system for implementing various embodiments of the present disclosure, according to embodiments.

FIG. 2 is a flowchart illustrating a method for well-being management in a living environment, according to embodiments.

FIG. 3 is a flowchart illustrating a method for well-being management in a living environment, according to embodiments.

FIG. 4 is a flowchart illustrating a method for well-being management in a living environment, according to embodiments.

FIG. 5 is a flowchart illustrating a method for well-being management in a living environment, according to embodiments.

FIG. 6 depicts a diagram of an example system for well-being management with respect to a living environment, according to embodiments.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the disclosure relate to cognitive adaptations for well-being management in a living environment. Disclosed aspects may utilize analytics and measurements from micro-cognitive systems to determine an anomalous state, and thereafter perform an action once the anomalous state is detected. Inputs from a plurality of micro cognitive systems may be received. The micro-cognitive systems may be configured to self-learn, identify a set of behavior patterns of an individual, and to trigger an alarm parameter in response to a pattern mismatch. The inputs received from the plurality of micro cognitive systems may be integrated to form integrated data by a central processing system. The integrated data may be analyzed to identify behavioral data and alarm parameters. Behavioral norms for a subject may be established using the behavioral data and the identified alarm parameters. Predetermined criteria may be used to determine whether an anomalous event has been detected, and a response action may be performed in response to determining the anomalous event. Leveraging self-learning micro-cognitive systems to detect anomalous events based on behavioral norms may be associated with personal well-being, event response efficiency, and quality of life.

Aspects of the disclosure relate to the recognition that, in some situations, individuals may face challenges associated with independent living. For instance, some individuals may face challenges related to walking/moving unassisted, preparing meals, remembering appointments or events, communicating with others, or the like. In such situations, it may be desirable to monitor the living environment and behavior of individuals to ascertain whether particular events or behaviors are in accordance with typical behavior patterns for that individual, or whether they may be indicative of an anomaly. Accordingly, aspects of the disclosure relate to utilizing analytics and measurements from micro-cognitive systems of a living environment to determine an anomalous state. In response to determining the anomalous state, an action may be performed to facilitate management or handling of the anomalous state. In this way, the well-being of individuals in independent living environments may be positively impacted.

Aspects of the disclosure include a system, method, and computer program product of cognitive adaptations (e.g., cognitive systems, micro cognitive systems) for well-being management in a living environment. A set of sensor-derived data for the living environment may be ingested. The ingestion of a set of sensor-derived data may occur using a set of micro-cognitive modules. The set of sensor-derived data may be analyzed using a machine learning technique. The set of sensor-derived data may be analyzed to detect an anomalous event related to the living environment. The anomalous event may be detected based on the set of sensor-derived data. An anomalous event response action may be performed in response to detecting the anomalous event.

In embodiments, a set of individualized sensor-derived norms may be generated with respect to an individual based on the set of sensor-derived data. A new sensor-derived entry may be received with respect to the individual. A comparison of the new sensor-derived data entry may be carried out with the set of individualized sensor-derived norms to identify a non-normative event. Based on the comparison achieving a threshold distinction, the non-normative event which indicates the anomalous event may be identified. In embodiments, a notification which indicates the anomalous event may be provided to perform the anomalous event response action. Altogether, aspects of the disclosure can have performance or efficiency benefits (e.g., wear-rate, service-length, reliability, speed, flexibility, load balancing, responsiveness, stability, high availability, resource usage, productivity). Aspects may save resources such as bandwidth, disk, processing, or memory.

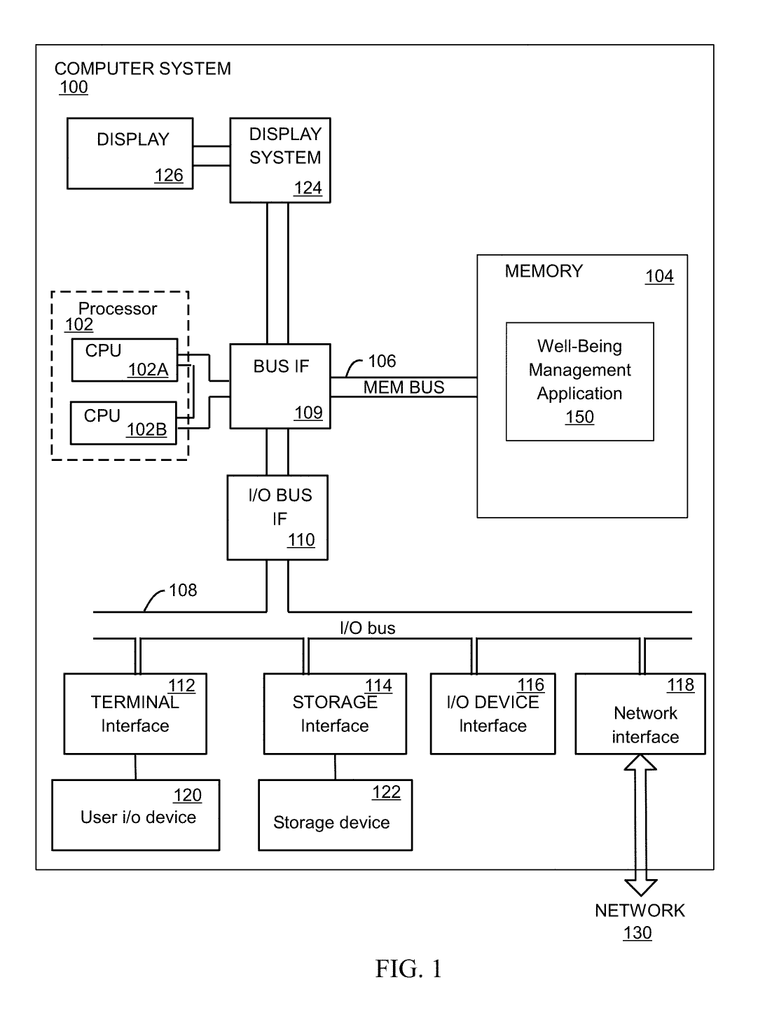

Turning now to the figures, FIG. 1 depicts a high-level block diagram of a computer system for implementing various embodiments of the present disclosure, according to embodiments. The mechanisms and apparatus of the various embodiments disclosed herein apply equally to any appropriate computing system. The major components of the computer system 100 include one or more processors 102, a memory 104, a terminal interface 112, a storage interface 114, an I/O (Input/Output) device interface 116, and a network interface 118, all of which are communicatively coupled, directly or indirectly, for inter-component communication via a memory bus 106, an 110 bus 108, bus interface unit 109, and an I/O bus interface unit 110.

The computer system 100 may contain one or more general-purpose programmable central processing units (CPUs) 102A and 102B, herein generically referred to as the processor 102. In embodiments, the computer system 100 may contain multiple processors; however, in certain embodiments, the computer system 100 may alternatively be a single CPU system. Each processor 102 executes instructions stored in the memory 104 and may include one or more levels of on-board cache.

In embodiments, the memory 104 may include a random-access semiconductor memory, storage device, or storage medium (either volatile or non-volatile) for storing or encoding data and programs. In certain embodiments, the memory 104 represents the entire virtual memory of the computer system 100, and may also include the virtual memory of other computer systems coupled to the computer system 100 or connected via a network. The memory 104 can be conceptually viewed as a single monolithic entity, but in other embodiments the memory 104 is a more complex arrangement, such as a hierarchy of caches and other memory devices. For example, memory may exist in multiple levels of caches, and these caches may be further divided by function, so that one cache holds instructions while another holds non-instruction data, which is used by the processor or processors. Memory may be further distributed and associated with different CPUs or sets of CPUs, as is known in any of various so-called non-uniform memory access (NUMA) computer architectures.

The memory 104 may store all or a portion of the various programs, modules and data structures for processing data transfers as discussed herein. For instance, the memory 104 can store a well-being management application 150. In embodiments, the well-being management application 150 may include instructions or statements that execute on the processor 102 or instructions or statements that are interpreted by instructions or statements that execute on the processor 102 to carry out the functions as further described below. In certain embodiments, the well-being management application 150 is implemented in hardware via semiconductor devices, chips, logical gates, circuits, circuit cards, and/or other physical hardware devices in lieu of, or in addition to, a processor-based system. In embodiments, the well-being management application 150 may include data in addition to instructions or statements.

The computer system 100 may include a bus interface unit 109 to handle communications among the processor 102, the memory 104, a display system 124, and the I/O bus interface unit 110. The I/O bus interface unit 110 may be coupled with the I/O bus 108 for transferring data to and from the various I/O units. The I/O bus interface unit 110 communicates with multiple I/O interface units 112, 114, 116, and 118, which are also known as I/O processors (IOPs) or I/O adapters (IOAs), through the I/O bus 108. The display system 124 may include a display controller, a display memory, or both. The display controller may provide video, audio, or both types of data to a display device 126. The display memory may be a dedicated memory for buffering video data. The display system 124 may be coupled with a display device 126, such as a standalone display screen, computer monitor, television, or a tablet or handheld device display. In one embodiment, the display device 126 may include one or more speakers for rendering audio. Alternatively, one or more speakers for rendering audio may be coupled with an I/O interface unit. In alternate embodiments, one or more of the functions provided by the display system 124 may be on board an integrated circuit that also includes the processor 102. In addition, one or more of the functions provided by the bus interface unit 109 may be on board an integrated circuit that also includes the processor 102.

The I/O interface units support communication with a variety of storage and I/O devices. For example, the terminal interface unit 112 supports the attachment of one or more user I/O devices 120, which may include user output devices (such as a video display device, speaker, and/or television set) and user input devices (such as a keyboard, mouse, keypad, touchpad, trackball, buttons, light pen, or other pointing device). A user may manipulate the user input devices using a user interface, in order to provide input data and commands to the user I/O device 120 and the computer system 100, and may receive output data via the user output devices. For example, a user interface may be presented via the user I/O device 120, such as displayed on a display device, played via a speaker, or printed via a printer.

The storage interface 114 supports the attachment of one or more disk drives or direct access storage devices 122 (which are typically rotating magnetic disk drive storage devices, although they could alternatively be other storage devices, including arrays of disk drives configured to appear as a single large storage device to a host computer, or solid-state drives, such as flash memory). In some embodiments, the storage device 122 may be implemented via any type of secondary storage device. The contents of the memory 104, or any portion thereof, may be stored to and retrieved from the storage device 122 as needed. The I/O device interface 116 provides an interface to any of various other I/O devices or devices of other types, such as printers or fax machines. The network interface 118 provides one or more communication paths from the computer system 100 to other digital devices and computer systems; these communication paths may include, e.g., one or more networks 130.

Although the computer system 100 shown in FIG. 1 illustrates a particular bus structure providing a direct communication path among the processors 102, the memory 104, the bus interface 109, the display system 124, and the I/O bus interface unit 110, in alternative embodiments the computer system 100 may include different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface unit 110 and the I/O bus 108 are shown as single respective units, the computer system 100 may, in fact, contain multiple I/O bus interface units 110 and/or multiple I/O buses 108. While multiple I/O interface units are shown, which separate the I/O bus 108 from various communications paths running to the various I/O devices, in other embodiments, some or all of the I/O devices are connected directly to one or more system I/O buses.

In various embodiments, the computer system 100 is a multi-user mainframe computer system, a single-user system, or a server computer or similar device that has little or no direct user interface, but receives requests from other computer systems (clients). In other embodiments, the computer system 100 may be implemented as a desktop computer, portable computer, laptop or notebook computer, tablet computer, pocket computer, telephone, smart phone, or any other suitable type of electronic device.

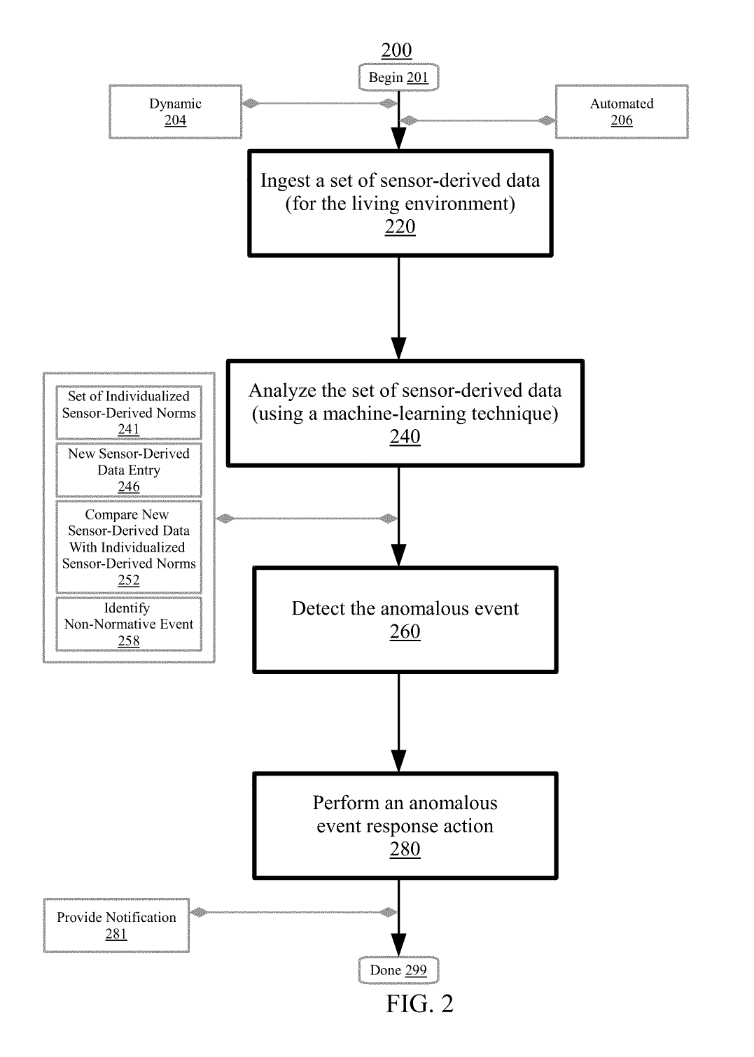

FIG. 2 is a flowchart illustrating a method 200 for well-being management in a living environment. Aspects of FIG. 2 relate to detecting an anomalous event based on a set of sensor-derived data for a living environment, and performing an anomalous event response action. Aspects of the disclosure relate to the relation that, in some situations, individuals (e.g., elderly individuals) may face challenges associated with independent living. Accordingly, aspects of the disclosure relate to utilizing analytics and data measured from micro-cognitive systems to determine an anomalous state with respect to a living environment, and performing a response action once an anomalous state is detected. The response action may positively impact the well-being of one or more individuals of the living environment. The living environment may include a setting inhabited by one or more individuals. For instance, the living environment may include a home environment (e.g., house, apartment), an assisted living environment (e.g., healthcare facility, assisted living facility, elder care facility), or other type of residence. In embodiments, the living environment may include an independent living environment in which an individual resides on their own. Other types of living environments are also possible. The method 200 may begin at block 201.

In embodiments, the ingesting, the analyzing, the detecting, the performing, and the other steps described herein may each occur in an dynamic fashion to streamline well-being management at block 204. For instance, the ingesting, the analyzing, the detecting, the performing, and the other steps described herein may occur in real-time, ongoing, or on-the-fly. As an example, one or more steps described herein may be performed in a dynamic fashion (e.g., a set of sensor-derived data for the living environment may be ingested and analyzed in real-time) in order to streamline (e.g., facilitate, promote, enhance) data packet management. Other methods of performing the steps described herein are also possible.

In embodiments, the ingesting, the analyzing, the detecting, the performing, and the other steps described herein may each occur in an automated fashion at block 206. In embodiments, the ingesting, the analyzing, the detecting, the performing, and the other steps described herein may be carried out by an internal well-being management module maintained in a persistent storage device of a local host node (e.g., micro-cognitive module) or locally connected hardware device (e.g., well-being engine). In embodiments, the ingesting, the analyzing, the detecting, the performing, and the other steps described herein may be carried out by an external well-being management module hosted by a remote computing device or server (e.g., accessible via a subscription, usage-based system, or other service model). In this way, aspects of well-being management may be performed using automated computing machinery without user intervention or manual action. Other methods of performing the steps described herein are also possible.

At block 220, a set of sensor-derived data for the living environment may be ingested. The set of sensor-derived data may be ingested using a set of micro-cognitive modules. Generally, ingesting can include receiving, importing, collecting, analyzing, transforming, processing, monitoring, or capturing the set of sensor-derived data using the set of micro-cognitive modules. The set of sensor-derived data may include information that describes, indicates, or otherwise characterizes the actions, activity, or behavior of an individual of the living environment. The set of sensor-derived data may include textual data (e.g., textual summaries or descriptions of environmental conditions or individual behavior), measurements (e.g., numbers, integers, statistics), audio data (e.g., captured recordings of sounds, voices), image data (e.g., pictures, still images, snapshots), video data (e.g., captured videos of actions or events) or the like. As examples the set of sensor-derived data may indicate the time that an individual got out of bed, the gait/walking speed of an individual, the frequency with which a user moves to a particular location (e.g., bathroom), the amount of food/water consumed by an individual in a given time period, sleep cycles (e.g., caloric intake), or other data or information that characterizes the behavior of an individual. In embodiments, the set of sensor-derived data may be ingested using a set of micro-cognitive modules. The set of micro-cognitive modules may include computing nodes configured to monitor one or more aspects of the living environment, and collect the set of sensor-derived data. In embodiments, the set of micro-cognitive modules may be configured to perform data analysis, machine learning, or cognitive computing techniques to examine the set of sensor derived data. The set of micro-cognitive modules may be configured in a communicative network such that data may be shared between multiple micro-cognitive modules of a living environment. In embodiments, the set of micro-cognitive modules may be communicatively connected to a well-being engine configured to aggregate and analyze the set of sensor-derived data ingested by each respective micro-cognitive module. In embodiments, ingesting the set of sensor-derived data may include configuring one or more micro-cognitive modules to use an array of sensors (e.g., motion sensors, biometric sensors, cameras, microphones) to collect data regarding a particular aspect of the living environment. As an example, a particular micro-cognitive module may be configured to capture data regarding the times that a refrigerator is opened by an individual. For instance, the micro-cognitive module may monitor the status of the refrigerator (e.g., open or closed), and determine that the refrigerator changes from a “closed” status to an “open” status at 10:14 AM. In embodiments, ingesting may include receiving direct input of data or information (e.g., by an authorized user, system administrator). As an example, a personal care physician may submit information regarding the nature of the medications taken by an individual. Other methods of ingesting the set of sensor-derived data for the living environment are also possible.

At block 240, the set of sensor-derived data may be analyzed using a machine learning technique. The set of sensor-derived data may be analyzed to detect an anomalous event related to the living environment. Generally, analyzing can include determining information regarding the content of the sensor-derived data (e.g., trigger condition, type of data). Analyzing can include examining (e.g., performing an inspection of the sensor-derived data), evaluating (e.g., generating an appraisal of the sensor-derived data), resolving (e.g., ascertaining an observation/conclusion/answer with respect to the sensor-derived data), parsing (e.g., deciphering structured and unstructured data constructs of the sensor-derived data), querying (e.g., asking a question regarding the sensor-derived data), or categorizing (e.g., organizing by a feature or type of the sensor-derived data). In embodiments, analyzing may include examining the set of sensor-derived data to extract properties or attributes that describe the living environment or characterize the behavior of an individual of the living environment. As an example, with respect to a set of sensor-derived data that indicates the times that an individual moves to a particular room (e.g., goes to the bathroom), analyzing may include calculating the frequency (e.g., number of times per day or week) that the individual moves to the particular room, the amount of time the individual spends in the particular room, or the like. As another example, with respect to a set of sensor-derived data including image data that indicates the facial expression of a user, analyzing may include using one or more image content analysis techniques to ascertain an emotion of the user that is indicated by the captured facial expression (e.g., happy, lonely, concerned). Other methods of analyzing the set of warning data are also possible.

In embodiments, aspects of the disclosure relate to analyzing the set of sensor-derived data using a machine-learning technique. The machine-learning technique may include one or more algorithms that allow computer systems to learn without being explicitly programmed. The machine learning technique may include a method of data analysis that automates analytical model building. The machine-learning technique may be configured to use algorithms that iteratively learn from data to recognize patterns, form deductions, and generate conclusions without being explicitly programmed to do so. The machine-learning technique may use computational statistics methods, predictive analytic methods, and pattern recognition techniques to identify trends in data, and generate models, relationship, hypotheses, and rules based on the identified trends. As examples, the machine-learning technique may include decision tree learning techniques (e.g., decision tree as a predictive model), associative rule-based learning techniques (e.g., to discover relations between variables), artificial neural networks (e.g., non-linear statistical data modeling tools to represent complex relationships between inputs and outputs, capture statistical structures in unknown joint probability distributions), deep learning techniques (e.g., modeling of high level abstractions in data using multiple processing layers including linear and non-linear transformations), inductive logic programming techniques (e.g., hypothesis derivation, logic programs that entails positive examples), support vector machines (e.g., classification and regression methods), clustering techniques (e.g., drawing observations from similar and dissimilar data structures), Bayesian networks (e.g., probabilistic graphic model that represents a set of random variables and their conditional independencies using a directed acyclic graph), reinforcement learning techniques (e.g., policy recognition that maps states to actions), representation learning techniques (e.g., discovering representations of inputs based on training), similarity and metric learning techniques (e.g., distinction between similar and dissimilar object pairs), sparse dictionary learning techniques (e.g., representing data as a linear combination of basis functions), rule-based machine learning techniques (e.g., method that identifies, learns, or evolves rules to store, manipulate, or apply knowledge), or learning classifier systems (e.g., context-dependency analysis). Other types of machine learning techniques are also possible.

In embodiments, aspects of the disclosure relate to analyzing the set of sensor-derived data using a machine-learning technique. In embodiments, analyzing the set of sensor-derived data using a machine-learning technique may include utilizing a density-based statistical clustering analysis technique to identify a set of data patterns based on the set of sensor-derived data. For instance, the clustering analysis technique may indicate that particular actions (e.g., eating breakfast) occur in close statistical relation with other actions (e.g., brushing teeth). Based on the set of data patterns identified by the density-based statistical clustering analysis, an associative rule-based technique may be applied to formulate a rule-based model relating the actions indicated by the set of data patterns. For instance, the associative-rule based technique may ascertain a causal relationship between the two actions of “eating breakfast” and “brushing teeth” (e.g., the individual brushes their teeth as a result of eating food). In embodiments, analyzing may include using an inductive logic programming technique to ascertain a set of candidate reasons (e.g., causes, triggers) that contextualize the set of sensor derived data. As an example, in response to identifying a data pattern that indicates that an individual is waking up 1-2 hours later in the morning than a previous establishing waking time, the inductive logic programming technique may examine the set of sensor-derived data to ascertain a potential reason for the later waking time (e.g., the individual has been going to bed later in the evening as a result of drinking a caffeinated beverage at night). Other methods of analyzing the set of sensor-derived data using the machine-learning technique are also possible.

In embodiments, a set of individualized sensor-derived norms may be generated with respect to an individual at block 241. The set of individualized sensor-derived norms may be based on the set of sensor-derived data. Generally, generating may include forming, computing, producing, calculating, deriving, formulating, or otherwise creating the set of individualized sensor-derived norms. The set of sensor-derived norms may include individualized benchmarks, baselines, valid activity ranges, or other parameters that characterize the behavior patterns of an individual of the living environment. For instance, the set of sensor-derived norms may include general rules that describe specific actions a particular individual takes at certain times, or how an individual responds to certain triggering events or other stimuli. As an example, the set of sensor-derived norms may include an established data pattern (e.g., indicated by the set of sensor-derived data) that indicates that an individual of the living environment typically wakes up between 6:10 and 6:30 AM, goes to the bathroom between 6:35 and 6:40 AM for a duration of 4 minutes, and eats breakfast between 7:00 and 7:30 AM, ingesting approximately 400 calories. In embodiments, generating the set of individualized sensor-derived norms may include ascertaining one or more actions that are routinely performed at a particular time, in a particular sequence, in response to a specific trigger, or the like. For instance, the set of sensor-derived data may be aggregated and analyzed to determine particular habits, routines, customs, or other trends indicated by the actions of an individual. As an example, a set of sensor-derived data for a period of time (e.g., one week, one month, one year) may be aggregated and examined to determine the days of the week and average time that an action of “dog walk” occurs for an individual, and an individualized sensor-derived norm of “the individual walks the dog at 3:45 PM on Mondays, Wednesdays, and Fridays” may be generated. Other methods of generating the set of individualized sensor-derived norms are also possible.

In embodiments, a new sensor-derived data entry may be received with respect to the individual at block 246. Generally, receiving can include detecting, sensing, collecting, gathering, capturing, ascertaining, or otherwise accepting delivery of the new sensor-derived data entry. The new sensor-derived data entry may include a recent, original, unknown, or additional portion of sensor-derived data. In embodiments, the new sensor-derived data entry may include a piece of sensor-derived data that was not included in the set of sensor-derived data (e.g., and thus may not have been used when generating the set of individualized sensor-derived norms). In embodiments, receiving may include capturing the new sensor-derived data entry subsequent to ingestion of the set of sensor-derived data. For instance, as described herein, the set of micro-cognitive modules may be configured to ingest the set of sensor-derived data for the living environment, and subsequently analyze the set of sensor-derived data using one or more machine learning techniques to generate the set of individualized sensor-derived norms for an individual of the living environment. Accordingly, subsequent to generation of the set of individualized sensor-derived norms, the set of micro-cognitive modules may capture an additional set of sensor-derived data that includes the new sensor-derived data entry. Other methods of receiving the new sensor-derived data entry are also possible.

In embodiments, a comparison of the new sensor-derived data entry may be carried-out at block 252. The new sensor-derived data entry may be compared with the set of individualized sensor-derived norms. The comparison of the new sensor-derived data entry with the set of individualized sensor-derived norms may occur to identify a non-normative event. Generally, carrying-out may include performing, implementing, instantiating, completing, or otherwise executing the comparison of the new sensor-derived data entry with the set of individualized sensor-derived norms. In embodiments, carrying-out the comparison may include examining the new sensor-derived data entry with respect to the set of individualized sensor-derived norms. For instance, the new sensor-derived data entry may be contrasted with respect to the set of individualized sensor-derived norms to determine whether or not the new-sensor derived data entry corresponds (e.g., matches, agrees with) an existing data pattern exhibited by the set of individualized sensor-derived norms. As an example, consider a situation in which a set of individualized sensor-derived norms indicates that an individual retrieves the mail from the mailbox every day between 3:00 and 4:00 PM. A new sensor-derived data entry that indicates that a user has not retrieved the mail from the mailbox by 9:00 PM in the evening may be detected. Accordingly, the new sensor-derived entry may be compared with the individualized sensor-derived norm pertaining to mail retrieval to determine whether the new sensor-derived entry corresponds with the set of individualized sensor-derived norms. In embodiments, in the event that the new sensor-derived data entry does not correspond with the set of individualized sensor-derived norms, a non-normative event may be detected. The non-normative event may include an action, lack of action, activity, occurrence, irregularity, or other happening that differs, deviates from, contradicts, diverges from, or is otherwise incongruous with the set of individualized sensor-derived norms. Other methods of carrying-out the comparison between the new sensor-derived data entry and the set of individualized sensor-derived norms to identify the non-normative event are also possible.

In embodiments, the non-normative event may be identified at block 258. The non-normative event may be identified based on the comparison achieving a threshold distinction. The non-normative event may indicate the anomalous event. Generally, identifying can include detecting, sensing, recognizing, ascertaining, discovering, or otherwise determining the non-normative event. Aspects of the disclosure relate to the recognition that, in some situations, new sensor-derived data entries may deviate to some degree from the set of individualized sensor-derived norms without being indicative of a non-normative event (e.g., eating dinner one hour later than a usual time may not represent an irregularity). Accordingly, aspects of the disclosure relate to identifying the non-normative event in response to the comparison between the new sensor-derived data entry and the set of individualized sensor derived norms achieving a threshold distinction. The threshold distinction may include a difference, discrepancy, or mismatch between the new sensor-derived data entry and the set of individualized sensor derived norms that exceeds a threshold. The threshold distinction may be calculated independently for each individualized sensor-derived norm based on the statistical data pattern exhibited by the set of sensor-derived data with respect to a corresponding action, event, or occurrence. As an example, for an individualized sensor-derived norm that indicates that an individual takes medication at 12:00 PM each day, the threshold distinction may include a 2 hour tolerance window before and after 12:00 PM (e.g., if the individual takes the medication between 10:00 AM and 2:00 PM, this may fall within the range of acceptable deviation). As described herein, identifying may include determining that the comparison achieves the threshold distinction. Consider, for example, an individualized sensor-derived norm which indicates that a first individual engages in a telephone call with a second individual (e.g., son or daughter) every day at 8:30 PM. The sensor-derived norm may be associated with a threshold distinction of 36 hours. Accordingly, a new sensor-derived data entry that indicates that 40 hours have passed without the first individual speaking on the phone with the second individual may be received and compared with the individualized sensor-derived norm. In embodiments, the comparison may achieve the threshold distinction (e.g., 40 hours achieves the 36 hour threshold distinction), and the non-normative event may be identified. Other methods of identifying the non-normative event are also possible.

At block 260, the anomalous event may be detected. The anomalous event may be detected based on the set of sensor-derived data. Generally, detecting can include sensing, recognizing, discovering, distinguishing, ascertaining, or otherwise determining the anomalous event. The anomalous event may include an inconsistency, deviation, abnormality, eccentricity, or other irregularity with respect to the set of sensor derived data ingested for a particular living environment (e.g., the set of individualized sensor-derived norms for an individual). The anomalous event may indicate a change to the living environment (e.g., such as the behavior of a user) that deviates from previous data patterns exhibited by the set of sensor-derived data. For instance, the anomalous event may indicate that an individual of the living environment is engaging in an activity that is not consistent with past behavior, or is not engaging in an action that he or she has historically performed. As examples, the anomalous event may include performing an action at a different time than normal (e.g., eating a first meal of the day late in the afternoon), engaging in an action a greater number of times than in the past (e.g., going to the bathroom 20 times a day as opposed to 10 times per day in the past), not performing an action that is typically performed (e.g., not walking a dog in the afternoon), moving in a different manner (e.g., using a walker or a cane rather than walking unassisted), or the like. In embodiments, detecting the anomalous event may include monitoring the living environment and ascertaining that a particular action or behavior of an individual of the living environment mismatches a data pattern exhibited by the set of sensor-derived data. Other methods of detecting the anomalous event based on the set of sensor-derived data are also possible.

At block 280, an anomalous event response action may be performed. The anomalous event response action may be performed in response to detecting the anomalous event. In embodiments, performing can include carrying-out, implementing, instantiating, completing, or otherwise executing the anomalous event response action. The anomalous event response action may include a process, activity, operation, movement, or other procedure performed to manage the anomalous event. For instance, the anomalous event response action may be configured to resolve (e.g., fix, correct), stabilize (e.g., bring into alignment, return to normal), limit (e.g., mitigate) the impact, or otherwise assist in handling of the anomalous event. As examples, the anomalous event response action may include an alert provided to a designated party, a preventative measure to avoid repeated occurrence of the anomalous event, a proposal to mitigate the anomalous event response action, a data transmission of the nature (e.g., cause, severity, triggering parameters) of the anomalous event, a solution to a problem indicated by the anomalous event, or other type of action. In embodiments, performing may include examining the nature of the anomalous event to determine an appropriate anomalous event response action from a list of candidate anomalous event response actions, and initiating the appropriate anomalous event response action with respect to the living environment. Consider the following example. An anomalous event may be detected that indicates that the movement of a user has decreased below a threshold level (e.g., the daily number of steps of the individual falls below 5000, walking speed has fallen below 1 meters per second). Accordingly, an appropriate anomalous event response action of “providing a cane to the individual” may be determined, and suggested to a designated party (e.g., family member, healthcare provider for the individual) for approval. In response to receiving approval, a cane may be automatically ordered (e.g., from an online shopping store) and shipped to the residence of the individual to assist the movement of the individual. Other methods of performing the anomalous even response action are also possible.

In embodiments, a notification may be provided at block 281. The notification may be provided to perform the anomalous event response action. The notification may indicate the anomalous event. Aspects of the disclosure relate to the recognition that, in some situations, conveying information regarding the nature of the anomalous event to a designated party (e.g., family member, healthcare provider) may positively impact well-being management with respect to an individual of a living environment. Accordingly, aspects of the disclosure relate to providing a notification to perform the anomalous event response action. Generally, providing can include sending, conveying, relaying, displaying, preventing, or otherwise transmitting the notification. The notification may include an alert, a phone call, a pager communication, text message, e-mail, alarm, social media message, request for personnel dispatch (e.g., emergency response team) or the like. In embodiments, the notification may include information regarding potential causes of the anomalous event, the severity of the anomalous event, suggested solutions or resolution actions for the anomalous event, or other information regarding the anomalous event. In embodiments, providing may include transmitting the notification to one or more pre-designated parties. In certain embodiments, the recipients of the notification may be determined based on the nature of the anomalous event (e.g., anomalous events associated with relatively lesser severity levels may be transmitted to family members, while anomalous events associated with relatively greater security levels may be transmitted to emergency personnel). Consider the following example. An anomalous event may be detected that indicates that a first individual has not yet exited a bedroom of the living environment by 10:00 AM (e.g., the set of sensor-derived data indicates that the user typically exits the bedroom by 7:30 AM). Accordingly, a notification may be generated that indicates that the first individual has not exited the bedroom potentially as a result of catching a cold. The notification may include a suggestion to a second individual (e.g., recipient of the notification) to bring cold medication and chicken-noodle soup to the first individual. In embodiments, the notification may be transmitted to a designated recipient including a son of the first individual. Other methods of providing a notification to indicate the anomalous event are also possible.

Consider the following example. A set of sensor-derived data for an individual of a living environment may be collected. The set of sensor-derived data may include data regarding when the individual becomes active each morning, the food consumption habits, routines, activities, movement patterns, and other information for the individual. The set of sensor-derived data may be analyzed using a machine-learning technique to generate a set of individualized sensor derived norms for the individual. As an example, data patterns of the set of sensor-derived data may be identified to recognize correlations and sequences of actions, and an individualized sensor-derived norm may indicate that the individual eats a midday meal between 1:00 and 1:30 PM each day, then brushes his or her teeth within 10 minutes of finishing the meal, and subsequently takes the dog for a walk within 20 minutes of brushing his or her teeth. In embodiments, on a particular day, a new sensor-derived data entry may be received that indicates that 30 minutes have passed since the individual brushed his or her teeth, but the individual has not left to take the dog on a walk. In embodiments, the new sensor-derived data entry may be compared with the individualized sensor-derived norm, and it may be determined that a single missed instance of walking the dog does not achieve a threshold distinction of 4 days (e.g., a single missed instance may be the result of inclement weather, unfavorable air temperatures, temporary tiredness, or other causes that may not be indicative of an anomalous event). The set of micro-cognitive modules may continue collecting sensor-derived data for the individual. In certain embodiments, a new sensor-derived data entry may be detected that 4 days have passed without the individual taking the dog for a walk. A comparison of the new sensor-derived data entry may be compared with the individualized sensor-derived norm, and it may be ascertained that the comparison achieves the threshold distinction of 4 days, such that an anomalous event may be detected with respect to the living environment. Accordingly, an anomalous event response action to manage the anomalous event may be performed. As an example, a notification may be sent to a designated individual (e.g., son or daughter of the individual) that indicates that the individual has not taken the dog on a walk for 4 days, which diverges from typical behavior patterns for the individual. Other methods of well-being management in a living environment are also possible.

Method 200 concludes at block 299. As described herein, aspects of method 200 relate to using a set of sensor-derived data to dynamically (e.g., in real-time, ongoing, on-the-fly) detect an anomalous event and to perform an anomalous event response action. Aspects of method 200 may provide performance or efficiency benefits for improving well-being in a living environment. As an example, an anomalous event indicating that the food consumption habits of an individual have deviated with respect to the typical food consumption habits of the individual may be detected based on a set of sensor-derived data, and an anomalous event response action of a notification may be provided to a designated individual to manage the anomalous event. Altogether, leveraging self-learning micro-cognitive systems to detect anomalous events based on behavioral norms may be associated with personal well-being, event response efficiency, and quality of life.

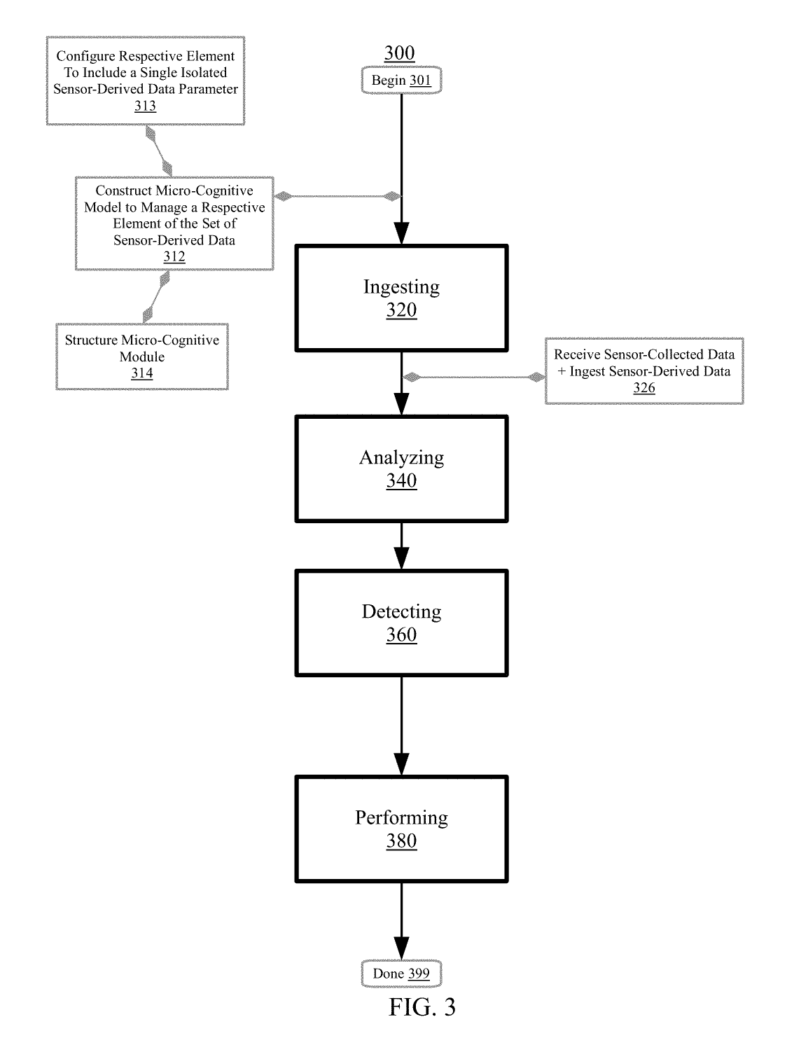

FIG. 3 is a flowchart illustrating a method 300 for well-being management in a living environment, according to embodiments. Aspects of FIG. 3 relate to configuring a set of micro-cognitive modules and a well-being engine to facilitate well-being management with respect to a living environment. Aspects of method 300 may be similar or the same as aspects of method 200, and aspects may be utilized interchangeably with one or more methodologies described herein. The method 300 may begin at block 301. At block 320, a set of sensor-derived data for the living environment may be ingested. The set of sensor-derived data may be ingested using a set of micro-cognitive modules. At block 340, the set of sensor-derived data may be analyzed using a machine learning technique. The set of sensor-derived data may be analyzed to detect an anomalous event related to the living environment. At block 360, the anomalous event may be detected. The anomalous event may be detected based on the set of sensor-derived data. At block 380, an anomalous event response action may be performed. The anomalous event response action may be performed in response to detecting the anomalous event. Altogether, leveraging self-learning micro-cognitive systems to detect anomalous events based on behavioral norms may be associated with personal well-being, event response efficiency, and quality of life.

In embodiments, a respective micro-cognitive module of the set of micro-cognitive modules may be constructed at block 312. The respective micro-cognitive module may be constructed to manage a respective element of the set of sensor-derived data. Generally, constructing can include assembling, building, programming, structuring, or otherwise configuring the respective micro-cognitive module to manage a respective element of the set of sensor-derived data. Aspects of the disclosure, in certain embodiments, relate to configuring each micro-cognitive module of the set of micro-cognitive modules to be responsible for monitoring, tracking, or otherwise managing a respective element of the set of sensor-derived data. The respective element may include a specific or particular aspect of the set of sensor-derived data. As examples, the respective element may include medication consumption times, refrigerator access frequency, gait monitoring, facial expression tracking, dietary monitoring, inter-personal communication detection, sleep pattern analysis, and the like. In embodiments, constructing may include structuring (e.g., designing, assembling, equipping) a particular micro-cognitive module with a set of sensors appropriate for monitoring of a particular aspect of the living environment, and installing the micro-cognitive module within the living environment so as to facilitate management of the particular aspect. As an example, for a particular aspect of “refrigerator access frequency,” a micro-cognitive module may be constructed to include a motion sensor, and positioned such that opening and closing of the refrigerator door is captured by the motion sensor. In certain embodiments, constructing the respective micro-cognitive module may include configuring the micro-cognitive module with a software application configured to perform analytics or processing operations on the captured set of sensor-derived data. For instance, the micro-cognitive module may be configured to calculate the frequency of refrigerator access based on the number of times the refrigerator door is opened and closed together with a corresponding time period. Other methods of constructing a micro-cognitive module to manage a respective element of the set of sensor-derived data are also possible.

In embodiments, configuration of the respective element of the set of sensor-derived data may occur at block 313. The respective element of the set of sensor-derived data may be configured to include a single isolated sensor-derived data parameter. Generally, configuring can include arranging, formulating, setting, programming, or otherwise organizing the respective element of the set of sensor-derived data to include a single isolated sensor-derived data parameter. The single isolated sensor-derived data parameter may include an individual or distinct feature, aspect, or property that is independent (e.g., separated, secluded) from other elements of the set of sensor-derived data. For instance, the single isolated sensor-derived data parameter may include a particular sub-portion that represents a discrete characteristic of the respective element of the set of sensor-derived data. As an example, for a respective element of “movement patterns,” the single isolated sensor-derived parameter may include a property of “walking speed.” As another example, for a respective element of “sleep cycle,” the single isolated sensor-derived parameter may include a property of “inhalation frequency while asleep.” In embodiments, configuring the respective element of the set of sensor derived data to include the single isolated sensor-derived data parameter may include breaking down a particular respective element into a plurality of sub-properties, and selecting a single sub-property as the single isolated sensor-derived data parameter (e.g., to be monitored, collected, or captured by a respective micro-cognitive module of the set of micro-cognitive modules). Other methods of configuring the respective element of the set of sensor-derived data to include the single isolated sensor-derived data parameter are also possible.

In embodiments, structuring of the respective micro-cognitive module may occur at block 314. Generally, structuring can include assembling, building, designing, constructing, or otherwise configuring the respective micro-cognitive module. In embodiments, the respective micro-cognitive module may be structured to include a data storage unit. The data storage unit may include computer component including recording media configured to retain digital data. As examples, the data storage unit may include a hard disk drive, random-access memory, a solid state drive, flash memory, memory cards, cloud storage, or the like. As an example, the data storage unit may include a 32 gigabyte flash memory device configured to collect and store the set of sensor-derived data. In embodiments, the respective micro-cognitive module may be structured to include a cognitive analytics module. The cognitive analytics module may include a computing component configured to perform one or more processing or analysis techniques with respect to the sensor-collected data. As examples, the cognitive analytics module may include a processing unit configured to perform cognitive computing, text analytics, deep learning, natural language processing, digital image/video processing, object recognition, or other type of analysis technique. For instance, the cognitive analytics module may include a statistical analysis technique configured to identify patterns with respect to the set of sensor-derived data. In embodiments, the respective micro-cognitive module may be structured to include an event generator. The event generator may include a computing module configured to identify key actions, occurrences, or happenings indicated by the set of sensor-derived data. As an example, the event generator may be configured to recognize a non-normative event that indicates a deviation or irregularity with respect to the set of sensor-derived data. For instance, the event generator may be configured to ascertain that an individual not wearing a coat when they go outside is indicative of a non-normative event for the individual. In embodiments, the respective micro-cognitive module may be structured to include an event handler. The event handler may include a computing module configured to resolve, regulate, govern, handle, or otherwise manage an event (e.g., a non-normative event detected by an event generator). As an example, the event handler may be configured to generate and transmit a notification to a designated individual in response to verifying that a non-normative event achieves a distinction threshold to indicate an anomalous event with respect to the set of sensor-derived data. Other methods of structuring the respective micro-cognitive module are also possible.

In embodiments, receiving and ingesting may occur at block 326. Aspects of the disclosure, in embodiments, relate to gathering raw data using the set of micro-cognitive modules, and performing analysis and processing operations on the data using a centralized well-being engine. In embodiments, a set of sensor-collected data may be received by the set of micro-cognitive modules. Generally, receiving can include detecting, sensing, collecting, monitoring, gathering, capturing, ascertaining, or otherwise accepting delivery of the set of sensor-collected data. The set of sensor-collected data may include raw, unrefined data and information collected by the set of sensors prior to analysis and processing (e.g., by the set of micro-cognitive modules or the well-being engine). In embodiments, receiving can include using a set of sensors (e.g., cameras, microphones, motion sensors, infrared sensors) to monitor and gather data regarding various aspects of the living environment. As an example, receiving may include using a pedometer to count the number of steps taken by an individual from a first point to a second point. In embodiments, the set of sensor-derived data may be ingested by a well-being engine. The ingesting of the set of sensor-derived data may occur in response to the ingesting using the set of micro-cognitive modules. Generally, ingesting can include importing, gathering, collecting, analyzing, aggregating, transforming, processing, or accumulating the set of sensor-derived data. In embodiments, the set of sensor-derived data may have undergone one or more preliminary processing operations to arrange, organize, or format the set of sensor-collected data into a form that may be readily interpretable by the well-being engine. The well-being engine may include a centralized processing module configured to aggregate the set of sensor-derived data from the micro-cognitive modules of the living environment, and perform one or more analytics operations (e.g., machine learning techniques) on the acquired data. In embodiments, ingesting may include importing the set of sensor-derived data from a storage device of each respective micro-cognitive module, and initiating performance of a deep learning technique to form hypotheses and draw conclusions about the sensor-derived data. In embodiments, ingesting may include receiving direct input of data or information (e.g., by an authorized user, system administrator). As an example, a personal care physician may submit information regarding the nature of the medications taken by an individual. In embodiments, the well-being engine may be configured to assemble a normative behavior model for an individual of the living environment that incorporates the properties, attributes, and indications of the sensor-derived data into a cohesive body of rules, knowledge, and deductions that may grow and evolve based on changes to the living environment. Other methods of receiving the set of sensor-collected data and ingesting the set of sensor-derived data are also possible. Method 300 may conclude at block 399.

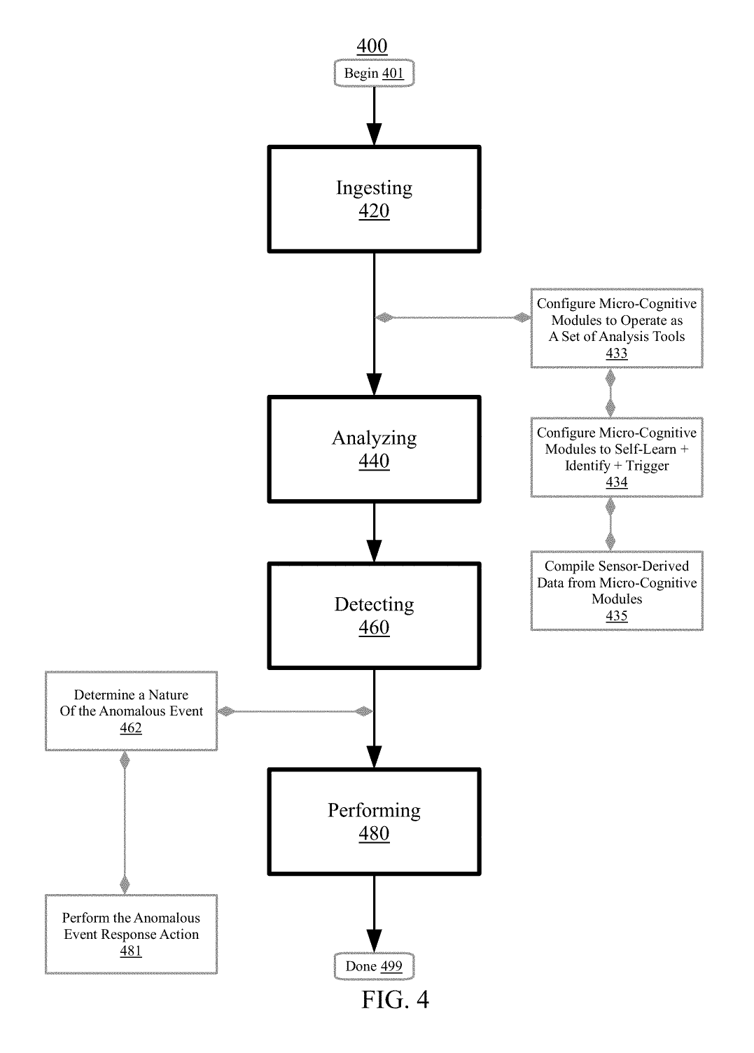

FIG. 4 is a flowchart illustrating a method 400 for well-being management in a living environment, according to embodiments. Aspects of FIG. 4 relate to configuring a set of micro-cognitive modules and managing an anomalous event. Aspects of method 400 may be similar or the same as aspects of method 200/300, and aspects may be utilized interchangeably with one or more methodologies described herein. The method 400 may begin at block 401. At block 420, a set of sensor-derived data for the living environment may be ingested. The set of sensor-derived data may be ingested using a set of micro-cognitive modules. At block 440, the set of sensor-derived data may be analyzed using a machine learning technique. The set of sensor-derived data may be analyzed to detect an anomalous event related to the living environment. At block 460, the anomalous event may be detected. The anomalous event may be detected based on the set of sensor-derived data. At block 480, an anomalous event response action may be performed. The anomalous event response action may be performed in response to detecting the anomalous event. Altogether, leveraging self-learning micro-cognitive systems to detect anomalous events based on behavioral norms may be associated with personal well-being, event response efficiency, and quality of life.

In embodiments, configuration of the set of micro-cognitive modules may occur at block 433. The set of micro-cognitive modules may be configured to operate as a set of analysis tools. The set of micro-cognitive modules may examine, in isolation, a single element of a measurable behavior of an individual. Generally, configuring can include assembling, programming, arranging, instructing, or otherwise setting-up the set of micro-cognitive modules to operate as a set of analysis tools to examine a single element of a measurable behavior of an individual. Aspects of the disclosure, in certain embodiments, relate to utilizing the set of micro-cognitive modules to perform processing and analysis operations (e.g., rather than a well-being engine). In embodiments, configuring may include structuring both a hardware feature and a software feature of one or more micro-cognitive modules to facilitate examination of a single element of the measurable behavior of an individual. For instance, a micro-cognitive module may be equipped with a particular type of sensor that is specifically adapted for collection of a certain type of data, as well as a software application specifically configured to analyze the collected data. As an example, a micro-cognitive module may be equipped with a photosensor configured to detect when light is turned on or off in a bedroom of the living environment (e.g., to detect when an individual becomes active or rests). The micro-cognitive module may include a software application configured to analyze the luminosity and duration of the detected light in order to determine whether a lamp has been voluntarily turned on by a user, lightning has flashed, or a nightlight has automatically activated. Other methods of configuring the set of micro-cognitive modules to operate as a set of analysis tools to examine a single element of a measurable behavior of an individual are also possible.

In embodiments, configuration of the set of micro-cognitive modules may occur at block 434. The set of micro-cognitive modules may be configured to self-learn. The set of micro-cognitive modules may identify a set of behavior patterns of an individual. The set of micro-cognitive modules may trigger an alarm parameter in response to a pattern mismatch. Generally, configuring can include assembling, programming, arranging, instructing, or otherwise setting-up the set of micro-cognitive modules. In embodiments, the set of micro-cognitive modules may include one or more machine-learning techniques to facilitate self-learning of the actions, behaviors, and events that relate to a particular living environment. In embodiments, self-learning may include extracting relationships from the set of sensor-derived data, and using the extracted relationships to define a set of rules that characterize the behavior of a user. New rules may be added to the set of rules as additional data regarding the behavior of an individual is collected. In embodiments, identifying a set of behavior patterns of an individual may include using a statistical analysis technique to identify particular actions that occur in close statistical relation with one another. As an example, the statistical analysis technique may recognize a plurality of actions that occur in a particular sequence that repeats periodically as a behavior pattern of an individual. In embodiments, triggering an alarm parameter in response to a pattern mismatch may include comparing a data point corresponding to a first action with an established behavior pattern, and ascertaining that the data point differs, deviates, or diverges from the established behavior pattern. Accordingly, in response to determining the mismatch, an alert or other type of notification may be generated and transmitted to a designated individual. Other methods of configuring the set of micro-cognitive modules to self-learn, identify a set of behavior patterns of an individual, and trigger an alarm parameter in response to a pattern mismatch are also possible.

In embodiments, the set of sensor-derived data may be compiled from the set of micro-cognitive modules at block 435. The set of sensor-derived data may be compiled by a well-being engine. The set of sensor-derived data may be in an integrated form in response to the compiling. Generally, compiling can include assembling, integrating, accumulating, editing, collecting, assimilating, aggregating, formatting, or otherwise organizing the set of sensor-derived data by the well-being engine. As described herein, the well-being engine may include a centralized processing module configured to aggregate the set of sensor-derived data from the micro-cognitive modules of the living environment, and perform one or more analytics operations (e.g., machine learning techniques) on the acquired data. In embodiments, the integrated form may include a data structure that incorporates, logs, archives, or otherwise records the set of sensor-derived data in an organized fashion. In embodiments, compiling may include filtering the set of sensor-derived data to remove information irrelevant information, combining like properties, categorizing by type or attribute, forming deductions/conclusions based on observations, or otherwise consolidating the set of sensor-derived data into a systemized format. As an example, compiling may include sorting the set of sensor-derived data, and grouping portions of data that achieve a similarity threshold (e.g., walking speed and number of steps, food consumption and refrigerator access). In embodiments, compiling may include merging or combining sensor-derived data pertaining to different aspects of the living environment (e.g., sleep patterns and food consumption) to draw conclusions and form deductions about the behavior of an individual. Other methods of integrating the set of sensor-derived data are also possible.

In embodiments, a nature of the anomalous event may be determined at block 462. The nature of the anomalous event may be determined using a predetermined criterion. Generally, determining can include resolving, deriving, computing, identifying, formulating, or otherwise ascertaining the nature of the anomalous event. The nature of the anomalous event may include a type, kind, quality, characteristic, or attribute of the anomalous event. As examples, the nature may include a cause, reason, severity, category, time-sensitiveness, or other property of the anomalous event. In embodiments, determining the nature of the anomalous event may be based on a predetermined criterion. The predetermined criterion may include a benchmark, principle, guideline, or rubric that links a particular property of the anomalous event with a corresponding nature. For instance, a plurality of different anomalous events may be grouped into the same category, and linked to a nature through the predetermined criterion. As an example, for an anomalous event of “round trip walking time between the living room and the kitchen has increased by 50%,” a predetermined criterion may link the anomalous event to a nature of “walking-related anomaly.” As examples, other types of natures for anomalous events may include “sleeping-related anomaly,” “eating-related anomaly,” or the like. Other methods of determining the nature of the anomalous event are also possible.

In embodiments, the anomalous event response action may be performed at block 481. The anomalous event response action may be performed based on the nature of the anomalous event. Aspects of the disclosure, in embodiments, relate to determining and performing an anomalous event response action specifically configured to manage a particular anomalous event. Generally, performing can include carrying-out, implementing, instantiating, completing, or otherwise executing the anomalous event response action based on the nature of the anomalous event. In embodiments, performing may include ascertaining an appropriate anomalous event response action based on the nature of the anomalous event. In certain embodiments, a respective nature of an anomalous event may be linked with a predetermined set of candidate anomalous event response actions associated with expected positive impacts with respect to resolving or managing the anomalous event. For instance, for an anomalous event associated with a nature of “walking-related anomaly” the nature may be associated with a candidate anomalous event response action of “notification to a personal care physician,” where an anomalous event associated with a nature of “emotion-related anomaly” (e.g., loneliness) may be associated with a candidate anomalous event response action of “notification to a son or daughter.” In embodiments, some natures of anomalous events may be associated with response actions of phone calls to designated users, some natures may be associated with response actions of text messages to designated users, and some natures may be associated with response actions of emails to designated users (e.g., based on the potential severity or harm caused by the anomalous event, the temporal-sensitivity of the anomalous event). As an example, in response to determining an anomalous event of “caloric intake of the user has decreased below a threshold level,” performing may include transmitting a report to a dietary care provider regarding describing the details of the anomalous event. Other methods of performing the anomalous event response action based on the nature of the anomalous event are also possible. Method 400 may conclude at block 499.

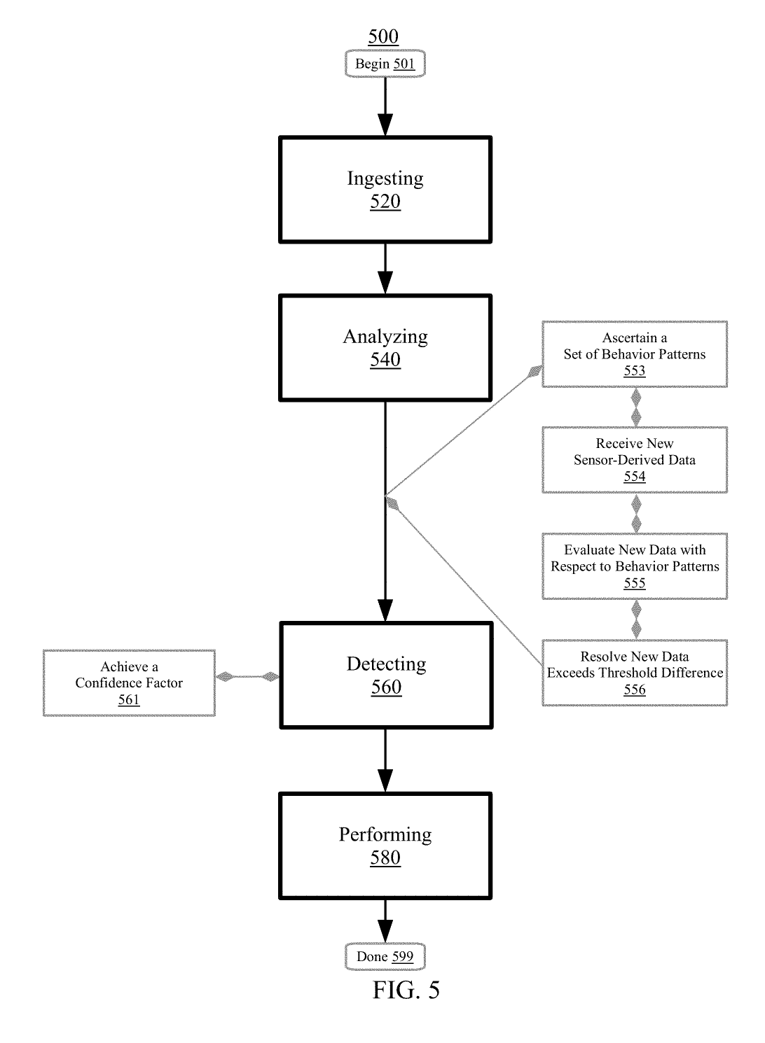

FIG. 5 is a flowchart illustrating a method 500 for well-being management in a living environment, according to embodiments. Aspects of FIG. 5 relate to resolving a new sensor-derived data entry with respect to a set of behavior patterns ascertained using a machine learning technique. Aspects of method 500 may be similar or the same as aspects of method 200/300/400, and aspects may be utilized interchangeably with one or more methodologies described herein. The method 500 may begin at block 501. At block 520, a set of sensor-derived data for the living environment may be ingested. The set of sensor-derived data may be ingested using a set of micro-cognitive modules. At block 540, the set of sensor-derived data may be analyzed using a machine learning technique. The set of sensor-derived data may be analyzed to detect an anomalous event related to the living environment. At block 560, the anomalous event may be detected. The anomalous event may be detected based on the set of sensor-derived data. At block 580, an anomalous event response action may be performed. The anomalous event response action may be performed in response to detecting the anomalous event. Altogether, leveraging self-learning micro-cognitive systems to detect anomalous events based on behavioral norms may be associated with personal well-being, event response efficiency, and quality of life.

In embodiments, a set of behavior patterns may be ascertained at block 553. The set of behavior patterns may be ascertained using the machine learning technique. The set of behavior patterns may be ascertained with respect to an individual. Generally, ascertaining may include resolving, deriving, computing, identifying, formulating, or otherwise determining the set of behavior patterns. The set of behavior patterns may include trends, tendencies, routines, customs or other sequences of actions or events that occur in relation to one another (e.g., repeat periodically, have a causal relationship). In embodiments, ascertaining the set of behavior patterns may include using a statistical analysis technique to identify particular actions that occur in close statistical relation with one another, and generating a set of associated rules to characterize/generalize the relation between the identified events. In embodiments, a new sensor-derived data entry may be received with respect to the individual at block 554. Generally, receiving can include detecting, sensing, collecting, gathering, capturing, ascertaining, or otherwise accepting delivery of the new sensor-derived data entry. In embodiments, receiving the new sensor-derived entry can include capturing a new portion of sensor-derived data subsequent to ingestion of the set of sensor-derived data. In embodiments, the new sensor-derived data entry may be evaluated with respect to the set of behavior patterns at block 555. Generally, evaluating can include analyzing, investigating, parsing, examining, or otherwise assessing the new sensor-derived data entry with respect to the set of behavior patterns. In embodiments, evaluating may include comparing the new sensor-derived data entry with the set of behavior patterns to ascertain a relative similarity or degree of divergence between the new sensor-derived data entry and the set of behavior patterns. In embodiments, the new sensor-derived data entry may be resolved to exceed a threshold difference with respect to the set of behavior patterns at block 556. Generally, resolving can include deriving, computing, identifying, ascertaining, formulating, or otherwise determining that the new sensor-derived data entry exceeds a threshold difference (e.g., threshold distinction) with respect to the set of behavior patterns. In embodiments, resolving may include ascertaining that the new sensor-derived data entry diverges from the set of behavior patterns by a pre-determined degree or extent. Other methods of ascertaining the set of behavior patterns, receiving a new sensor-derived data entry, evaluating a new sensor-derived data entry, and resolving that the new sensor-derived data entry exceeds a threshold difference are also possible.

In embodiments, a confidence factor may be achieved at block 561. The confidence factor may be achieved to trigger detection of the anomalous event. The confidence factor may be achieved with respect to the set of sensor-derived data. Generally, achieving may include accomplishing, attaining, fulfilling, satisfying, obtaining, or otherwise meeting the confidence factor. The confidence factor may include a quantitative representation, expression, or indication of the degree to which a determination regarding an anomalous event is considered to be correct, reliable, or accurate. As examples, the confidence factor may include a likelihood, weighting value, or probability. In embodiments, the confidence factor may be expressed as an integer between 0 and 100, where lesser values indicate lesser confidence and greater values indicate greater confidence that a determination regarding an anomalous event is correct. In embodiments, achieving the confidence factor may include comparing a non-normative event (e.g., potential anomalous event) to a set of evaluation criteria that assess the likelihood that the non-normative event is sufficiently divergent from the individualized sensor-derived norms/behavior patterns to be considered an anomalous event. For instance, achieving the confidence factor may include ascertaining that a threshold number of criteria (e.g., 3 criteria, 50% of all the criteria) are satisfied, identifying that one particular criterion is satisfied to a degree greater than a threshold value (e.g., criterion satisfaction over 80%), determining that a certain criterion that is weighted as highly impactful is not met, computing a statistical improbability that the non-normative event is a false positive, computing a statistical probability that the non-normative event is accurate, or the like.

As an example, consider a situation in which a non-normative event of “an individual does not walk his or her dog for 5 days” is detected. The non-normative event may be analyzed, and a plurality of potential causes/reasons for the non-normative event may be determined (e.g., the dog's leash has been lost, inclement weather has prevented the individual from going outside, the individual has a cold, the individual is experiencing a walking problem, another individual is walking the dog). In embodiments, the plurality of potential causes may be verified using the set of sensor-derived data to ascertain a likelihood that it is accurate. The likelihoods of each potential cause may be weighed against one another to compute a confidence factor (e.g., overall statistical likelihood that the non-normative event qualifies as an anomalous event). As an example, a confidence factor of “64%” may be determined for the non-normative event. In certain embodiments, the confidence factor may be compared with respect to a confidence factor threshold (e.g., 60%). In response to achieving the confidence factor threshold, the non-normative event may be identified as an anomalous event. Other methods of achieving the confidence factor are also possible. Method 500 concludes at block 599.

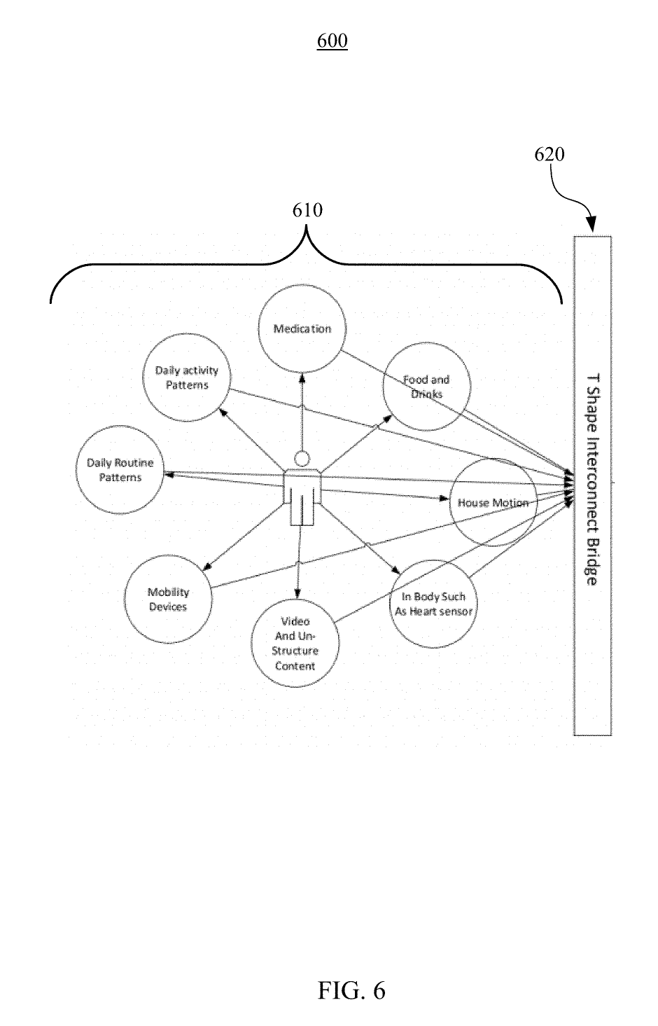

FIG. 6 depicts a diagram of an example system 600 for well-being management with respect to a living environment, according to embodiments. In embodiments, the example system 600 may include an array of sensors 610 connected to a T-shape interconnector bridge 620. Each sensor of the array of sensors 610 may be configured to monitor a particular aspect of the living environment and collect a set of sensor-derived data. As examples, sensors of the array of sensors 610 may be configured to monitor the medication consumption, food and drink consumption, movement patterns, biometric data (e.g., pacemaker), video and unstructured content, mobility devices (e.g., walkers, canes), daily routines, daily activity patterns, and the like. As additional examples, sensors of the array of sensors 610 may include patterns of movement from room to room, step count per unit time (e.g., steps per day, step speed), path deviation (e.g., step hesitancy), food consumption patterns (e.g., refrigerator-access sensors), biometric sensors (e.g., remote or on-body sensing of body temperature, heart rate, blood pressure, respiration), bodily waste analysis, audible patterns (e.g., talking, humming to oneself), steadiness (e.g., accelerometers, gyroscopes, visual sensors), sleep and rest patterns, schedules and appointment compliances (e.g., leaving the living environment at a particular time for an appointment, performing a calendar event), facial expressions, or the like. In embodiments, the T-shape interconnector bridge 620 may include a well-being management engine configured to aggregate, process, and perform one or more analysis operations on the set of sensor-derived data. In embodiments, the T-shape interconnector bridge 620 may be configured to detect an anomalous event based on the set of sensor-derived data, and perform an anomalous event response action to manage the anomalous event. Other types of systems for well-being management are also possible.

Consider the following example. Aspects of the disclosure, in certain embodiments, relate to combining one or more types of sensor-derived data to form hypotheses and draw conclusions about the behavior of an individual. For instance, in certain embodiments, a set of sensor-derived data including a calendar (e.g., indicating appointments and events), food consumption habits (e.g., types of food and drink consumed, caloric intake), medication information (e.g., the types of prescribed pills and medications), sleeping habits (e.g., hours slept each night) and body weight information may be collected for an individual. In embodiments, the set of sensor-derived data may be analyzed to generate an individualized sensor-derived norm that indicates that the individual goes to a personal care physician each Monday morning, consumes an average of 1700 calories a day, takes 2 different pills twice a day (morning and evening), sleeps an average of 7 hours a night, and has an average body weight of 168 pounds that varies by 3 (e.g., based on the diet and activity of the individual over a short-term period of time). In embodiments, a new sensor-derived data entry may be detected that indicates that the body weight of the individual has decreased from 168 pounds to 164 pounds over a one-week period. The decrease in the body weight of the individual may be identified as a non-normative event. In embodiments, analysis techniques may be performed on the set of sensor-derived data, and it may be ascertained that a new medication was prescribed for the individual by the personal care physician. The nature of the new medication may be analyzed (e.g., based on a captured image of the medication label, direct information entry to the system by the physician or other authorized user), and it may be ascertained that the new medication may be associated with weight-loss. As such, the weight loss of the individual may be determined to be in accordance with the set of sensor-derived data (e.g., non-anomalous). In certain embodiments, a new sensor-derived data entry may be detected that indicates that the sleeping duration of the individual has decreased from 7 hours a night to 5.5 hours a night. A note from a personal care physician associated with the new medication may be analyzed, and it may be detected that insomnia is a potential side-effect of the new medication, and that the personal care physician should be contacted if insomnia is experienced. Accordingly, the decrease in the sleeping duration of the individual may be detected as an anomalous event, and an anomalous event response action of transmitting an indication to the personal care physician may be performed. Other methods of well-being management are also possible.

In addition to embodiments described above, other embodiments having fewer operational steps, more operational steps, or different operational steps are contemplated. Also, some embodiments may perform some or all of the above operational steps in a different order. The modules are listed and described illustratively according to an embodiment and are not meant to indicate necessity of a particular module or exclusivity of other potential modules (or functions/purposes as applied to a specific module).

In the foregoing, reference is made to various embodiments. It should be understood, however, that this disclosure is not limited to the specifically described embodiments. Instead, any combination of the described features and elements, whether related to different embodiments or not, is contemplated to implement and practice this disclosure. Many modifications and variations may be apparent to those of ordinary skill in the art without departing from the scope and spirit of the described embodiments. Furthermore, although embodiments of this disclosure may achieve advantages over other possible solutions or over the prior art, whether or not a particular advantage is achieved by a given embodiment is not limiting of this disclosure. Thus, the described aspects, features, embodiments, and advantages are merely illustrative and are not considered elements or limitations of the appended claims except where explicitly recited in a claim(s).

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Embodiments according to this disclosure may be provided to end-users through a cloud-computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud-computing resources are provided to a user on a pay-per-use basis, where users are charged only for the computing resources actually used (e.g., an amount of storage space used by a user or a number of virtualized systems instantiated by the user). A user can access any of the resources that reside in the cloud at any time, and from anywhere across the Internet. In context of the present disclosure, a user may access applications or related data available in the cloud. For example, the nodes used to create a stream computing application may be virtual machines hosted by a cloud service provider. Doing so allows a user to access this information from any computing system attached to a network connected to the cloud (e.g., the Internet).

Embodiments of the present disclosure may also be delivered as part of a service engagement with a client corporation, nonprofit organization, government entity, internal organizational structure, or the like. These embodiments may include configuring a computer system to perform, and deploying software, hardware, and web services that implement, some or all of the methods described herein. These embodiments may also include analyzing the client's operations, creating recommendations responsive to the analysis, building systems that implement portions of the recommendations, integrating the systems into existing processes and infrastructure, metering use of the systems, allocating expenses to users of the systems, and billing for use of the systems.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing is directed to exemplary embodiments, other and further embodiments of the invention may be devised without departing from the basic scope thereof, and the scope thereof is determined by the claims that follow. The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

The terminology used herein is for the purpose of describing particular embodiments only and is not intended to be limiting of the various embodiments. As used herein, the singular forms “a,” “an,” and “the” are intended to include the plural forms as well, unless the context clearly indicates otherwise. “Set of,” “group of,” “bunch of,” etc. are intended to include one or more. It will be further understood that the terms “includes” and/or “including,” when used in this specification, specify the presence of the stated features, integers, steps, operations, elements, and/or components, but do not preclude the presence or addition of one or more other features, integers, steps, operations, elements, components, and/or groups thereof. In the previous detailed description of exemplary embodiments of the various embodiments, reference was made to the accompanying drawings (where like numbers represent like elements), which form a part hereof, and in which is shown by way of illustration specific exemplary embodiments in which the various embodiments may be practiced. These embodiments were described in sufficient detail to enable those skilled in the art to practice the embodiments, but other embodiments may be used and logical, mechanical, electrical, and other changes may be made without departing from the scope of the various embodiments. In the previous description, numerous specific details were set forth to provide a thorough understanding the various embodiments. But, the various embodiments may be practiced without these specific details. In other instances, well-known circuits, structures, and techniques have not been shown in detail in order not to obscure embodiments.

what is claimed is:

1. A computer-implemented method of cognitive adaptations for well-being management in a living environment, the method comprising:
ingesting, using a set of micro-cognitive modules, a set of sensor-derived data for the living environment;
analyzing, using a machine learning technique, the set of sensor-derived data to detect an anomalous event related to the living environment;
detecting, based on the set of sensor-derived data, the anomalous event; and
performing, in response to detecting the anomalous event, an anomalous event response action.

2. The method of claim 1, further comprising:
generating, with respect to an individual, a set of individualized sensor-derived norms based on the set of sensor-derived data;
receiving, with respect to the individual, a new sensor-derived data entry;
carrying-out a comparison of the new sensor-derived data entry with the set of individualized sensor-derived norms to identify a non-normative event; and
identifying, based on the comparison achieving a threshold distinction, the non-normative event which indicates the anomalous event.

3. The method of claim 1, further comprising:
providing, to perform the anomalous event response action, a notification which indicates the anomalous event.

4. The method of claim 1, further comprising:
constructing a respective micro-cognitive module of the set of micro-cognitive modules to manage a respective element of the set of sensor-derived data.

5. The method of claim 4, further comprising:
configuring the respective element of the set of sensor-derived data to include a single isolated sensor-derived data parameter.

6. The method of claim 4, further comprising:
structuring the respective micro-cognitive module to include:
a data storage unit,
a cognitive analytics module,
an event generator, and
an event handler.

7. The method of claim 1, further comprising:
receiving, by the set of micro-cognitive modules, a set of sensor-collected data; and
ingesting, by a well-being engine in response to the ingesting using the set of micro-cognitive modules, the set of sensor-derived data.

8. The method of claim 1, further comprising:
configuring the set of micro-cognitive modules to operate as a set of analysis tools to examine, in isolation, a single element of a measurable behavior of an individual.

9. The method of claim 8, further comprising:
configuring the set of micro-cognitive modules to self-learn, to identify a set of behavior patterns of an individual, and to trigger an alarm parameter in response to a pattern mismatch.

10. The method of claim 9, further comprising:
compiling, by a well-being engine, the set of sensor-derived data from the set of micro-cognitive modules, wherein the set of sensor-derived data is in an integrated form in response to the compiling.

11. The method of claim 10, further comprising:
determining, using a predetermined criterion, a nature of the anomalous event.

12. The method of claim 11, further comprising:
performing, based on the nature of the anomalous event, the anomalous event response action.

13. The method of claim 1, further comprising:
achieving, to trigger detection of the anomalous event, a confidence factor with respect to the set of sensor-derived data.

14. The method of claim 1, further comprising:
ascertaining, using the machine learning technique, a set of behavior patterns with respect to an individual;
receiving, with respect to the individual, a new sensor-derived data entry;
evaluating the new sensor-derived data entry with respect to the set of behavior patterns; and
resolving that the new sensor-derived data entry exceeds a threshold difference with respect to the set of behavior patterns.

15. The method of claim 1, wherein the ingesting, the analyzing, the detecting, and the performing each occur in a dynamic fashion to streamline well-being management.

16. The method of claim 1, wherein the ingesting, the analyzing, the detecting, and the performing each occur in an automated fashion without user intervention.

17. The method of claim 2, further comprising:
constructing a respective micro-cognitive module of the set of micro-cognitive modules to manage a respective element of the set of sensor-derived data;
structuring the respective micro-cognitive module to include:
a data storage unit,
a cognitive analytics module,
an event generator, and
an event handler;
configuring the respective element of the set of sensor-derived data to include a single isolated sensor-derived data parameter;
receiving, by the set of micro-cognitive modules, a set of sensor-collected data;
ingesting, by a well-being engine in response to the ingesting using the set of micro-cognitive modules, the set of sensor-derived data;
achieving, to trigger detection of the anomalous event, a confidence factor with respect to the set of sensor-derived data; and
providing, to perform the anomalous event response action, a notification which indicates the anomalous event.
